(19) United States
(12) Patent Application Publication
(10) Pub. No.: US 2021/0369294 A1
(43) Pub. Date: Dec. 2, 2021

(54) Catheter System for Explanting an Intracardiac Pacing System

(71) Applicant: BIOTRONIK SE & Co. KG, Berlin (DE)

(72) Inventor: Brian M. TAFF, PORTLAND, OR (US)

(73) Assignee: BIOTRONIK SE & Co. KG, Berlin (DE)

(21) Appl. No.: 17/285,512

(22) PCT Filed: Sep. 19, 2019
(86) PCT No.: PCT/EP2019/075148

§ 371 Date: Apr. 15, 2021

Related U.S. Application Data
(60) Provisional application No. 62/746,570, filed on Oct. 17, 2018.

(51) Int. Cl. A61B 17/3205

(52) U.S. Cl. CPC A61B 17/32053; A61B 2017/320052

(57) ABSTRACT

An apparatus for explanting an intracardiac medical device that comprises a casing, a proximal end and a distal end comprising an anchor element, wherein the intracardiac medical device is anchored to a heart tissue of a patient via the anchor element, wherein heart tissue adheres to the casing. The apparatus comprises a protector device that extends along an extension direction and is configured to surround the casing, when the protector device is positioned at the casing. Further, the apparatus comprises an alignment device configured to align the protector device and the casing with each other, when the alignment device engages the intracardiac medical device. The apparatus also comprises a shearing element. The shearing element is configured to move along the casing, when the protector device is moved along the casing in a moving direction towards the distal end, for shearing off heart tissue adhered to the casing.

### CROSS-REFERENCE TO RELATED APPLICATIONS

This application is the United States national phase under 35 U.S.C. § 371 of PCT International Patent Application No. PCT/EP2019/075148, filed on Sep. 19, 2019, which claims the benefit of U.S. Patent Application Ser. No. 62/746,570, filed on Oct. 17, 2018, the disclosures of which are hereby incorporated by reference herein in their entireties.

### TECHNICAL FIELD

The present disclosure relates to an apparatus for explanting an intracardiac medical is device.

Particularly, the present disclosure relates to a shearing-tip-enabled catheter system for explanting an anatomically-encapsulated intracardiac pacing system, e.g. a leadless pacemaker.

### BACKGROUND

An intracardiac medical device can be an intracardiac pacing system, e.g. a leadless pacemaker. A leadless pacemaker is an artificial cardiac pacemaker which is of small size such that it can directly be placed within a patient's heart, in particular an atrium or a ventricle. Therefore, such a device does not need a pacing lead. A leadless pacemaker can be implanted into the heart's blood volume via a catheter.

The leadless pacemaker comprises an energy source, e.g. a battery. Depending on the properties of the battery, the leadless pacemaker can remain in the patient's heart for years.

Present market offerings for bradycardia support are increasingly pointing attention to the reduced, overall patient care risk profile touted by leadless pacemaker systems as compared to traditional, pocket-based formats. This revised support modality employs small, self-contained pulse generators that are anchored (e.g. via tine-based structures) within the heart's blood volume to administer therapy. Such designs have, thus far, leaned on the use of primary cell power support and nominal targeted service times of around 10 years.

Given their long-duration residence within the patient's body, it is common for auto-immune responses to motivate anatomical encapsulation of, at least portions of, the implanted device. In other words, this means that due to the device's long-term residence within the patient's anatomy, tissue can adhere to the intracardiac medical device. This encapsulation response challenges the clinical capacity for device explanation at the end of service as simple lasso/snare-based catheter systems cannot readily combat the increased device/physiology entanglement as compared to acute retrieval/explanation needs.

Presently, companies that offer implantable leadless pacer systems have provided tooling support for device implantation and, at best, marginal or configurable support for acute device retrieval. Such acute device support typically leans on the use of the implantation catheter or the adaptation of such a system through its pairing with compatible retrieval snares. In scenarios serviced by such acute device support, no gross autoimmune patient response is involved which means there are no substantially confounding anatomical conditions in effect to mandate compensatory procedural manipulations and/or mechanical interactions with the implant. As such, managing the device encapsulation associated with chronically implanted conditions, has been left out of scope for catheter-based systems made available by leadless device manufacturers and thus represents an undersupported need.

This undersupport for chronic device explanation has arisen as a nearer term concern than many in the leadless pacer market might have hoped due to battery complications that have shortened product lifetimes. In some cases, the product lifetimes have been reduced to less than half of the nominal 10 year duration a reduction sufficient enough to allow for encapsulation, creating special needs for appropriate management.

There is no consensus on how to best manage implants once their primary cells are no longer able to provide therapy. Some clinicians have discussed leaving the devices in place and simply installing additional devices to enable replacement support therapies. Others have pointed to the possible use of acute explanation tools in cases where device encapsulation (in a given patient) is not severe.

In cases where an expired implant is not removed from the patient's body and additional devices are installed to provide replacement therapy, the patient accumulates an increasing quantity of in-body hardware as a function of time. This added hardware can interfere with the nominal operation of the heart through modified compliance of the heart tissue, and reductions in the overall functional heart chamber blood volume. As a result of the limited flexibilities available for placing long cylindrical devices in but a handful of locations in support of viable interfacing with the patient's conduction system, optimal placement of subsequent implants additionally proves challenging. Such conditions can lead to the further progression of disease states, compromised oxygenation of tissues in the periphery, and the need for higher pacing thresholds (and hence shorter service times) in subsequent implants. Further, it cannot be guaranteed that multiple devices “banging into one another” in the heart will not create complications for the new device, cause anchoring erosion, or other possible deleterious effects.

Using acute explanation catheters to try and perform chronic explanation demands alignment with optimal patient conditions. Such an approach is only viable if the patient's auto-immune response does not motivate substantial encapsulation. There is no known means to improve the likelihood that a patient would not have an encapsulation response. In addition, there is no readily known method for determining a device encapsulation state when the therapy support needs replacing. Such a shortcoming typically means that the clinician has to access the patient's vasculature and then “try out” the acute explanation tooling in hopes that they are lucky enough to have found a patient where gross implant encapsulation is not in effect. The present disclosure is directed toward overcoming one or more of the above-mentioned problems, though not necessarily limited to embodiments that do.

### SUMMARY

Given this context, there is a strong need to provide more robust system support for chronic explanation (i.e. removal) of intracardiac medical devices to better address the management of tissue adhesion/attachment. This need is directly serviced by the apparatus described in claim 1 and the method described in claim 14. Embodiments are stated in the corresponding sub claims and are described below.

A first aspect is related to an apparatus for explanting an intracardiac medical device that comprises a casing, a proximal end and a distal end comprising an anchor element, wherein the intracardiac medical device is anchored to a heart tissue of a patient via the anchor element, wherein heart tissue adheres to the casing. The apparatus comprises a protector device (e.g. a protector cup) that extends along an extension direction. The protector device configured to surround the casing, when the protector device is positioned at the casing. Further, the apparatus comprises an alignment device, wherein the alignment device is configured to align the protector device and the casing with each other, when the alignment device engages the intracardiac medical device. The apparatus also comprises a shearing element. The shearing element is configured to move along the casing, when the protector device is moved along the casing in a moving direction towards the distal end, for shearing off heart tissue adhered to the casing.

An intracardiac medical device can be a leadless pacemaker. It is also referred to as implant throughout the present disclosure.

In an embodiment, the protector device comprises a shell delimiting the protector device in a circumferential direction. The circumferential direction can extend perpendicular to the extension direction of the protector device. The protector device can comprise an entrance orifice located at a distal end of the protector device. The entrance orifice can extend in a plane that extends perpendicular to the extension direction of the protector device.

The protector device can be moved along the casing in a moving direction. The moving direction can extend parallel to the extension direction of the protector device.

When the protector device is moved along the casing, there can be a gap between the shell of the protector device and the casing of the intracardiac medical device. A volume between the shell and the casing is also referred to as shielded zone throughout the present application.

The shearing element can shear off tissue adhered to the casing when the protector device is moved along the casing. Hence, the intracardiac medical device is released from adhered tissue such that the retention of the intracardiac medical device by tissue adhered to the casing is reduced advantageously. This can provide an easier explanation of the intracardiac medical device. “Shear off” is also referred to as “scrape off” throughout the present application.

Particularly, according to a preferred embodiment, the apparatus is a catheter system.

Particularly, the present disclosure provides a revised catheter system that enables a means for separating the device from surrounding encapsulation responses, addressing the shortcomings (at least for partially encapsulated implants) of presently available systems and improving support for explanation throughout the product lifecycle.

According to an embodiment, the shearing element comprises a cutting surface. Particularly, the cutting surface can have a conical shape.

Furthermore, in an embodiment, the cutting surface extends in a cutting plane. The shearing element can be configured such that there is an obtuse angle between the cutting plane and the casing, when the protector device is moved along the casing in a moving direction.

According to a further embodiment, the shearing element comprises a cutting edge. The cutting edge can comprise the most distal point of the shearing element. This means that the cutting edge can be the tip of the shearing element. The cutting edge can extend in a plane extending perpendicular to the extension direction of the protector device.

Further, according to an embodiment, the shearing element is positioned or positionable at a distal end of the protector device.

The shearing element can be positioned such that along the extension direction, the cutting surface is close to the distal end of the protector device. In particular, the shearing element can be positioned such that along the extension direction, the cutting surface is above the distal end of the protector device. This means that along the extension direction, the protector device extends beyond the shearing element, such that the protector device can is protect the shearing element, in particular the cutting surface.

Particularly, in an embodiment, the shearing element extends along a circumferential direction of the protector device.

Further, according to an embodiment, the cutting edge extends along the circumferential direction of the protector device.

According to a further embodiment, the shearing element is configured such that it can shear off tissue adhered to the casing in one working step, when the protector device is moved along the casing in the moving direction.

Particularly, in an embodiment, the shearing element is positioned inwards the protector device in a radial direction.

The protector device can comprise a longitudinal axis that extends along the extension direction. The radial direction can be positioned perpendicular to the longitudinal axis of the protector device and can point away from that axis, i.e. can point outwards. The radial direction can extend perpendicular to the extension direction of the protector device.

Furthermore, according to an embodiment, the shearing element is configured and arranged such that the cutting edge is positioned inwards the protector device in the radial direction. This means that in the radial direction, the protector device extends beyond the shearing element. In such an embodiment, the protector device advantageously protects the shearing element, in particular the cutting surface.

According to an embodiment, the shearing element comprises a cutting edge, wherein the shearing element is configured such that the cutting edge is distant to the casing, when the protector device is moved along the casing in a moving direction.

Further, the cutting edge can comprise the most distal point of the shearing element. This means that the cutting edge can be the tip of the shearing element.

The phrase “the cutting edge is distant to the casing” particularly means that there is a gap between the cutting edge and the casing. In particular, the cutting edge is distant to the casing in the radial direction.

According to a further embodiment, the shearing element comprises a contact section that is configured to contact the casing, when the protector device is moved along the casing in a moving direction. The shearing element shearing element can comprise a curved section configured such that the cutting edge is distant to the casing, when the protector device is moved along the casing in a moving direction.

When the cutting edge is distant to the casing when the protector device is moved along the casing, a probability is advantageously decreased that the cutting edge cuts into the casing, when the protector device is moved along the casing. Consequently, a probability to damage the intracardiac medical device during the shearing off of adhered tissue by the shearing element is decreased.

According to an embodiment, the apparatus is configured to press the shearing element against the casing, when the protector device is moved along the casing in the moving direction.

Furthermore, in an embodiment, the contact section of the shearing element is pressed against the casing, when the protector device is moved along the casing.

Particularly, when the shearing element is pressed against the casing (when the protector device is moved along the casing), the cutting surface can be brought in close proximity to tissue adhered to the casing. In particular, it can be brought in close proximity to a layer of the tissue that directly adheres the casing. This provides that the apparatus can shear off tissue adhered to the casing without a leftover which is still attached to the casing.

In an embodiment, the shearing element is spring mounted at the protector device such that is the shearing element presses against the casing, when the protector device is moved along the casing in the moving direction.

According to a further embodiment, the shearing element comprises at least one recess to increase a flexibility of the shearing element.

Furthermore, according to an embodiment, the shearing element comprises a plurality of recesses. In the circumferential direction, the recesses of the plurality of recesses can be arranged equally spaced to each other.

Further, in an embodiment, the cutting surface comprises at least one recess. Particularly, the recess can be configured to increase the flexibility of the shearing element in the radial direction.

In an embodiment, the recess is configured to adapt a diameter (a radius) of the shearing element, when the protector device is moved along the casing. Therefore, the shearing element can compensate for different sizes of the casing, in particular different diameters of the casing, while the shearing element can still be configured to press against the casing.

Furthermore, in an embodiment, the apparatus comprises at least one orifice configured to direct sheared-off heart tissue away from the casing.

According to a further embodiment, the protector device comprises at least one orifice. The shell of the protector device can comprise at least one orifice.

Further, according to an embodiment, the at least one orifice is configured to direct sheared-off heart tissue away from the shielded zone. The orifice can be configured such that it directs the sheared-off tissue outwards. This means that the sheared-off tissue that is directed away from the casing via the orifice is outwards the protector device in the radial direction. This advantageously prevents that the apparatus gets entangled in sheared-off tissue. This advantageously facilitates an undisturbed movement of the protector device along the moving direction. When sheared-off tissue is directed radially outwards the is protector device, this can also prevent that the sheared-off tissue is pushed downwards, in particular to prevent that the sheared-off tissue is compressed.

In an embodiment, the apparatus, in particular the protector device comprises a plurality of orifices.

According to an embodiment, the apparatus comprises a cleavage element, configured to cleave sheared-off heart tissue along the moving direction, when the protector device is moved along the casing in a moving direction.

In an embodiment, the apparatus comprises a cleavage element, configured to guide the sheared-off heart tissue towards the at least one orifice.

In an embodiment, the apparatus comprises a plurality of cleavage elements. In an embodiment, a cleavage element is positioned between two adjacent orifices. Particularly, the cleavage element can comprise a cutting surface, configured to cleave the sheared-off tissue.

Another aspect is related to a method for explanting an intracardiac medical device that comprises a casing, a proximal end and a distal end comprising an anchor element, wherein the intracardiac medical device is anchored to a heart tissue of a patient via the anchor element, wherein heart tissue adheres to the casing. An apparatus for explanting an intracardiac medical device according to the present disclosure is provided. The method comprises the steps of:
engaging the intracardiac medical device with the alignment device such that the protector device and the casing are aligned with each other, and
moving the protector device along the casing in a moving direction towards the distal end of the intracardiac medical device, and shearing off heart tissue adhered to the casing.

According to an embodiment, the apparatus can be aligned to the intracardiac medical device. The protector device can be moved along the casing in the moving direction and is tissue adhered to the casing of the intracardiac medical device can be sheared off by the shearing element of the apparatus.

Hence, by shearing-off adhered tissue, the intracardiac medical device is released from adhered tissue such that a retention of the intracardiac medical device by adhered tissue is reduced advantageously such that the explanation of the intracardiac medical device is easier than in a case in that tissue is adhered to the casing.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, further features, advantages and embodiments are explained with reference to the Figures, wherein

FIG. 1 to FIG. 10 illustrate the functioning of an embodiment of the apparatus for explanting an intracardiac medical device. Side views are presented. In brief:

FIG. 1 shows an initial situation in that an intracardiac medical device is anchored in the tissue of a patient;

FIGS. 2 to 4 illustrate the steps associated with engagements between the apparatus' alignment device and the intracardiac medical device;

FIGS. 5 to 8 show the positioning of the apparatus and a movement of the apparatus along a moving direction, and

FIGS. 9 and 10 show the release of the intracardiac medical device.

FIG. 11 shows the shearing element of the apparatus,

FIG. 12 shows a detailed view of FIG. 11,

FIG. 13 shows the distal end of the apparatus as viewed from below,

FIG. 14 shows a cleavage element and adjacent orifices of the apparatus,

FIG. 15 shows an orifice of the apparatus, and

FIG. 16 shows heart tissue after the explanation of an intracardiac medical device (as viewed from the eye perspective shown in FIG. 10).

### DETAILED DESCRIPTION

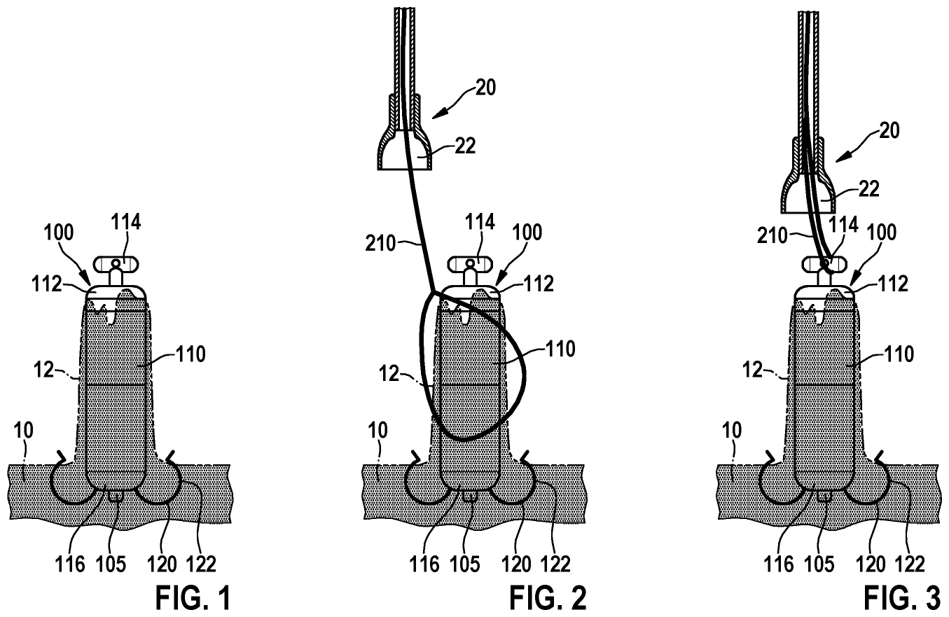

An embodiment of the intracardiac medical device 100 is shown that is anchored in the tissue 10 of a patient, in particular heart tissue 10. The illustrated intracardiac medical device 100 comprises a casing 110 and a proximal end 112 comprising a retrieval element 114. A distal end 116 of the intracardiac medical device 100 can comprise an electrode 105. Further, an anchor element 120 can be located at the distal end 116. The anchor element 120 can comprise a tine 122, in particular a plurality of tines 122. Via the tine 122 the intracardiac medical device 100 can be anchored in the tissue 10 (FIG. 1).

The intracardiac medical device 100 can be encapsulated by tissue 10. In other words this means that tissue 10 can adhere to the casing 110 of the intracardiac medical device 100. This tissue 10 is also referred to as adhered tissue 12 or encapsulation 12 throughout the present application.

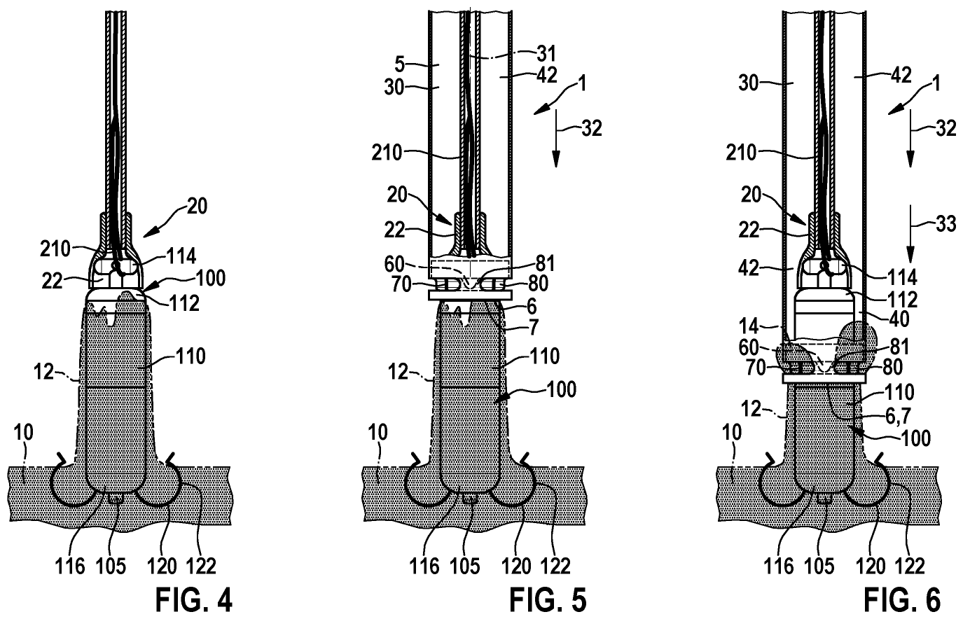

Via a lasso 210, an alignment device 20 can be directed towards the intracardiac medical device 100. The lasso 210 can attach to the retrieval element 114 (FIG. 2, FIG. 3). When aligned, the alignment device 20 can be positioned such that an alignment cup 22 of the alignment device 20 engages the retrieval element 114 (FIG. 4).

By means of the alignment device 20 the apparatus 1 can be aligned to the intracardiac medical device 100. In particular, a protector device 30 and the intracardiac medical device 100 can be aligned to each other. When aligned, the protector device 30 can be moved over the alignment cup 22 to the intracardiac medical device 100 (FIG. 5).

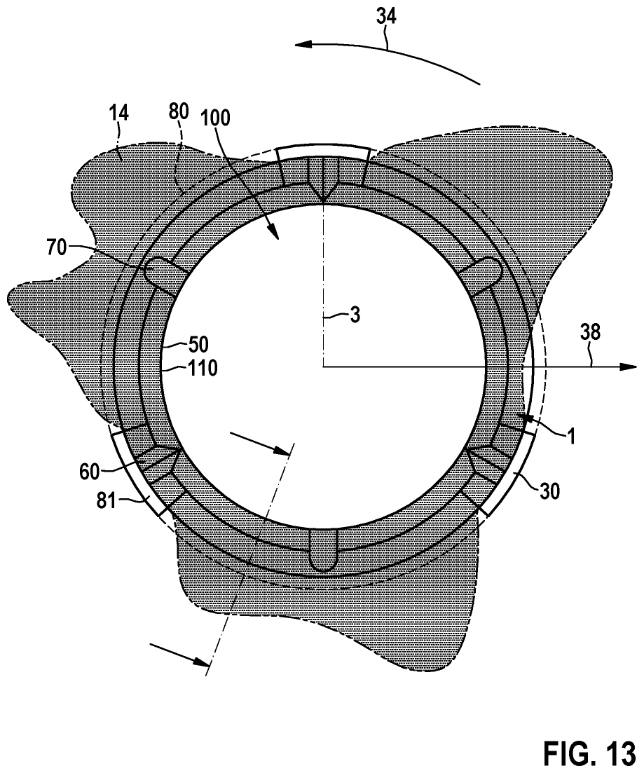

The protector device 30 can extend along an extension direction 32 (e.g. FIG. 5). The protector device can comprise a longitudinal axis 31 extending along the extension direction 32 (see FIG. 5). Perpendicular to the extension direction 32 the protector device 30 can have a circular cross section (see also FIG. 13). The protector device 30 can comprise a shell 42. The shell 42 can delimit the protector device 30 in the circumferential direction 34. The shell 42 can enclose an interior 5.

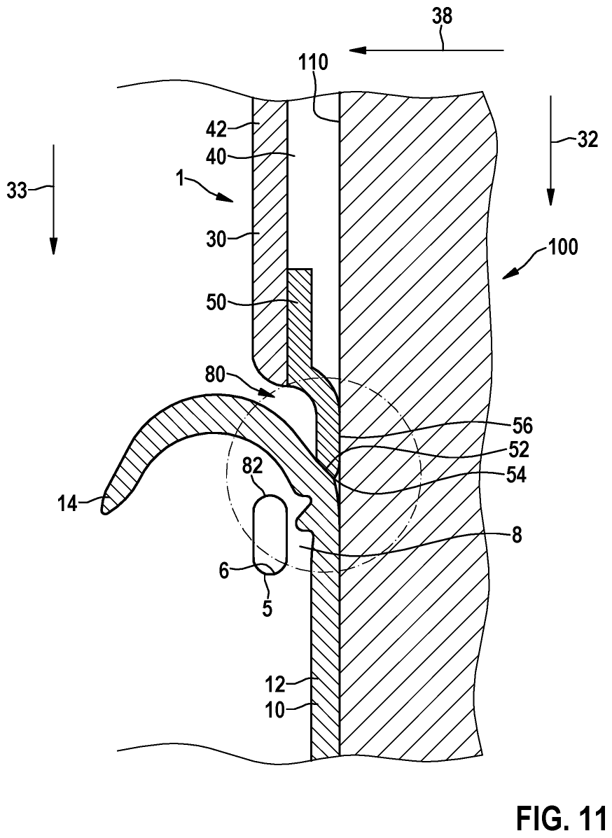

The protector device 30 can comprise a distal end 6. At the distal end 6, the protector device 30 can comprise an edge 7 that delimits an entrance orifice 80 of the protector device 30 through that the interior 5 can be accessed (see also FIG. 11).

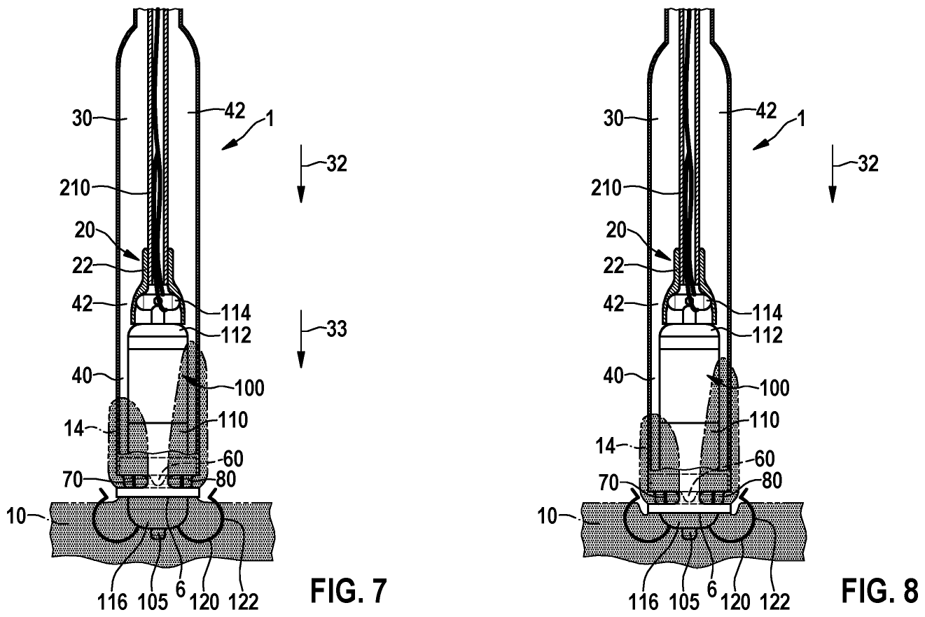

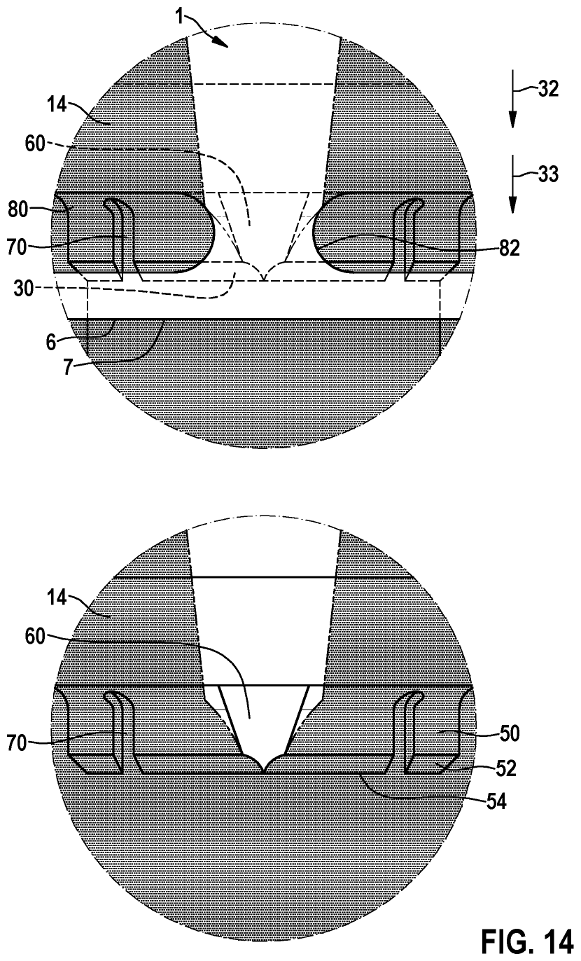

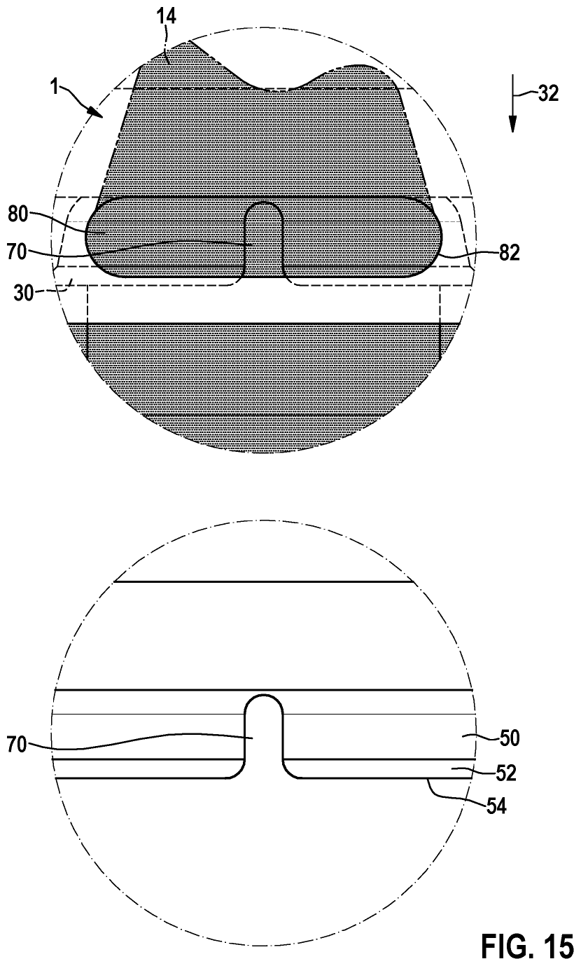

Close to the distal end 6, the protector device 30 can comprise an orifice 80, in particular a plurality of orifices 80. The orifice 80 can be configured to direct sheared-off tissue 14 away from the casing 110. In particular, the orifice 80 can be configured to direct sheared-off tissue 14 away from the shielded zone 40 (see FIG. 6-FIG. 9, FIG. 11, FIG. 14, FIG. 15). In the present embodiment, in the extension direction 32, each orifice 80 is located such that it has the same distance to the distal end 6 of the protector device 30. In the circumferential direction 34, two adjacent orifices 80 can be separated by a rib 81.

The protector device 30 can be positioned at the proximal end 112 of the intracardiac medical system 100 (FIG. 5) and can be moved along the moving direction 33 along the casing 110 towards the distal end 116 of the intracardiac medical system 100 (FIGS. 6-8). The shielded zone 40 can be located between the protector device 30, in particular an inner wall of the protector device 30 and the casing 110.

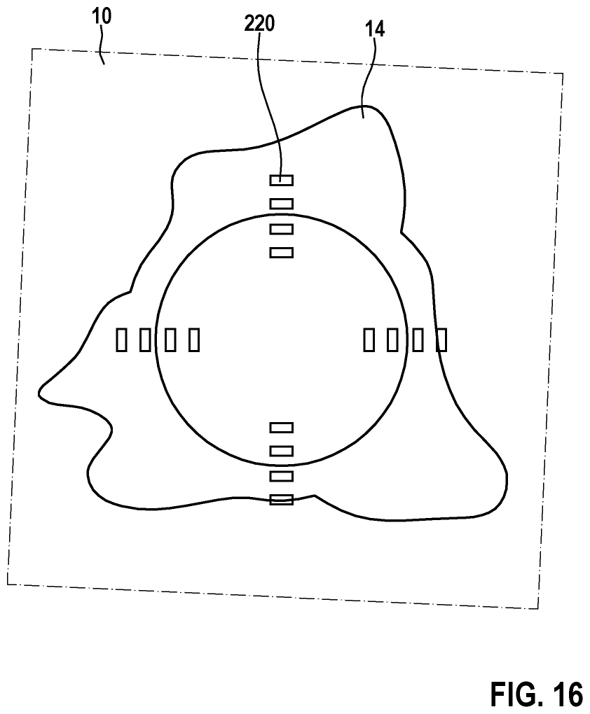

When moved along the moving direction 33, a shearing element 50 of the apparatus 1 (see also FIG. 15, FIG. 16) can shear off adhered tissue 12 which becomes sheared-off tissue 14. is Sheared-off tissue 14 is sheared off of the casing 110 but remains in connect to the tissue 10 (see FIG. 10).

In the present embodiment, the apparatus 1 comprises a cleavage element 60. The sheared-off tissue 14 can be cleaved in direction of the moving direction 33 by the cleavage element 60, when the protector device 30 is moved in the moving direction 33 along the casing 110. The cleavage element 60 can be configured to guide the sheared-off tissue 14, in particular cleaved sheared-off tissue 14, towards an adjacent orifice 80. By means of a cleavage element 60 and an adjacent orifice 80, sheared-off tissue 14 can be directed away from the shielded zone 40. In other words this means that by means of a cleavage element 60 and an adjacent orifice 80, sheared-off tissue 14 can be guided outwards, i.e. outside of the protector device 30, in particular outside the protector device 30 in a radial direction 38 (see also FIG. 11).

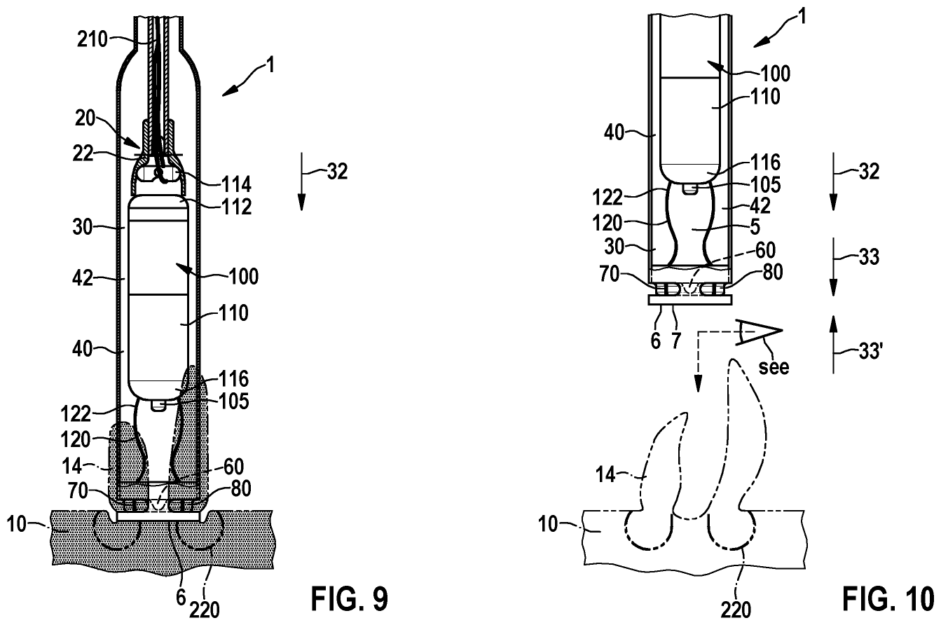

When the distal end 6 of the protector device 30 reaches the distal end 116 of the intracardiac medical device 100 (FIG. 8), the anchor element 120 can be released from the tissue 10. The intracardiac medical device 100 can be removed from the tissue 10 (FIG. 9).

In particular, the intracardiac medical device 100 can be moved in a direction opposite to the moving direction 33.

The removed intracardiac medical device 100 can be located in the interior 5 of the apparatus 1. In particular, the protector device 30 can surround the intracardiac medical device 100 removed from the heart tissue 10 (FIG. 9, FIG. 10). When the intracardiac medical device 100 is removed from the tissue 10, the protector device 30 and the intracardiac medical device 100 can be moved away from the tissue 10 in a removing direction 33′ that can be directed opposite to the moving direction 33.

When the apparatus 1 and the intracardiac medical device 100 are moved along the removing direction 33′, sheared-off tissue 14 can move through the respective orifices 80 such that it remains connected to the tissue 10 while the apparatus 1 and the intracardiac medical device 100 are removed from the tissue 10 (FIG. 10).

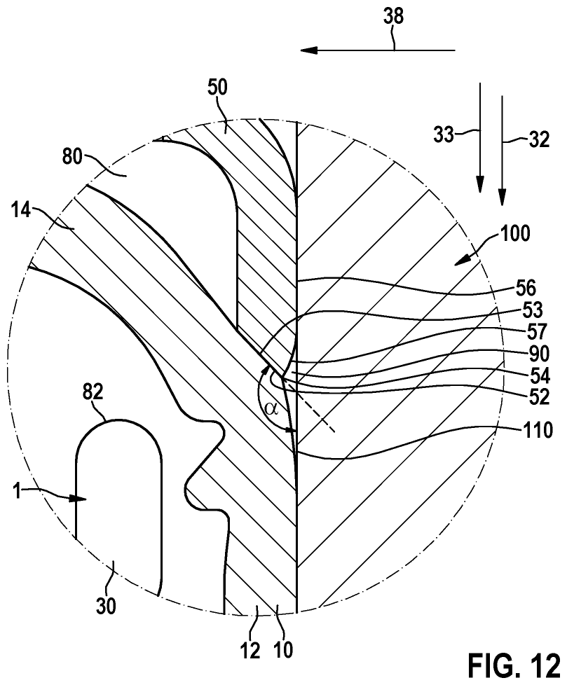

FIG. 11 and FIG. 12 illustrate the shearing element 50 in more detail, wherein FIG. 12 is an enlarged detailed view of FIG. 11.

FIGS. 11 and 12 show a cross-sectional side view of the apparatus 1 and the intracardiac medical device 100. A position of the apparatus 1 is presented in that one part of tissue 10 is adhered tissue 12 (i.e. tissue attached to the casing 110) and another part of the tissue 10 is sheared-off tissue 14. Sheared-off tissue 14 is directed towards an orifice 80 away from the intracardiac medical device 100. In particular, the sheared-off tissue 14 is directed away from the casing 110 in the radial direction 38.

The apparatus 1 can comprise a protector device 30 comprising an orifice 80 and a shearing element 50.

The shearing element 50 can be connected to the protector device 30. In an alternative embodiment, the protector device 30 comprises the shearing element 50.

In an embodiment, the shearing element 50 comprises a cutting surface 52. The cutting surface 52 can extend in a cutting plane 53. The shearing element 50 can be configured such that the cutting plane 53 and the casing 110 are at an obtuse angle a to each other, when the protector device 30 is moved along the casing 110 in the moving direction 33.

The shearing element 50 can comprise a cutting edge 54. In an embodiment, the cutting edge 54 comprises the most distal point of the shearing element 50. The cutting edge 54 can be a tip of the shearing element 50.

The shearing element 50 can be configured and located such that in the radial direction 38 the cutting element 50 is located inwards the protector device 30. In particular, the shearing element 50 can be configured such that the cutting edge 54 is located radially inwards the protector device 30.

In an embodiment, the shearing element 50 is configured and located such that along the extension direction 32 the distal end 6 of the protector device 30 is more distal than the cutting edge 54. In other word this means that along the extension direction 32, the protector device 30 extends beyond the cutting edge 54 such that the cutting edge 54 is located inside the protector device 30.

The shearing element 50 can comprise a contact section 56. In an embodiment, the shearing element 50 comprises a curved section 57. In an embodiment, the curved section 57 is arranged between the contact section 56 and the cutting edge 54.

The shearing element 50 can be configured such that the cutting edge 54 is distant to the casing 110 in the radial direction 38, when the shearing element 50, in particular the contact section 56 of the shearing element 50, is pressed against the intracardiac medical device 100. This means that in the radial direction 28, a gap 90 between the cutting edge 54 and the casing 110 can exist while the contact section 56 of the shearing element 50 can contact the casing 110 (FIG. 12). In particular, the curved section 57 is curved such that the cutting edge 54 is radially (i.e. in the radial direction 38) distant to the casing 110, when the protector device 30 is moved along the casing 110 in the moving direction 33.

The orifice 80 can be delimited by an edge 82 of the orifice 80. In an embodiment, the edge 82 of the orifice 80 can be rounded. A rounded edge 82 of the orifice 80 can decrease a probability to harm or grab sheared-off tissue 14 when it passes through the orifice 80.

In FIG. 13, the distal end 6 of an embodiment of the apparatus 1 is illustrated. The protector device 30 can have a circular shape extending in a circumferential direction 34. The protector device 30 and the intracardiac medical device 100 can be aligned coaxially.

The protector device 30 can comprise a plurality of orifices 80. In an embodiment, the protector device 30 comprises three orifices 80. The plurality of orifices 80 can be located equally spaced to each other in the circumferential direction 34. This means that in the circumferential direction 34 the distance between two adjacent orifices 80 is equal. A rib 81 can be positioned between two adjacent orifices 80 (along the circumferential direction 34).

In an embodiment, each orifice 80 of the plurality of orifices 80 has the same shape. In an embodiment, each orifice 80 has the same size.

In the circumferential direction 34, a cleavage element 60 can be arranged between two adjacent orifices 80.

The shearing element 50 can comprise a recess 70. In an embodiment, a shearing element 50 comprises a plurality of recesses. According to an embodiment, the number of recesses 70 equals the number of orifices 80. In an embodiment, the shearing element 50 is arranged such that the recess 70 and an orifice are arranged one above the other in a radial direction 38.

An embodiment of the cleavage element 60 and an embodiment of the orifice 80 are presented in FIG. 14 and FIG. 15.

The cleavage element 60 can be wedge-shaped. In an embodiment, the cleavage element 60 is arranged between two adjacent recesses 70 (FIG. 14). When the apparatus 1, in particular when the protector device 30, is moved along the moving direction 33, the cleavage element 60 can cleave sheared-off tissue 14. In an embodiment, the cleavage element 60 is configured to guide the cleaved sheared-off tissue 14 towards a respective adjacent orifice 80 (FIG. 14, FIG. 15).

The shearing element 50 can comprise a recess 70 (FIG. 15). A recess 70 (in particular the plurality of recesses 70) can be configured to increase the flexibility of the shearing element 50 in the radial direction 38. In particular, in an embodiment a radius of the shearing element 50 is not fixed but can adapt according to the radius 3 of the intracardiac medical device 110.

FIG. 16 illustrates a top view of tissue 10 after the removal of the intracardiac medical is device and the apparatus (perspective pointed to through the eye feature in FIG. 10). Tissue 10 and connected sheared-off tissue 14 is shown. A plurality of dotted lines 220 is shown. The dotted lines 220 show tine pathways within the tissue which are below the surface of the tissue. A dotted line 220 indicates a position at that the tissue was penetrated by a tine, when the intracardiac medical device is anchored in the tissue.

In one embodiment, a catheter-based system 1 with a guarded shearing element 50 at its distal tip 6 is provided. This shearing element 50 is used to scrape or shear off encapsulation 12 that surrounds the main-body capsule 110 of the leadless implant 100. Such shearing occurs as a result of the suite of procedures one would use for acute devices recapture (i.e. cinching a lasso 210 about the implant's hitch 114, placing a protector cup 30 over the implant's body 110, and withdrawing the deployed tines 122 back into an un-deployed state). This sequence is shown in the cascade of events shown in FIGS. 1-5 (implant 100 recapture) and continued into FIGS. 6-10 (encapsulation 12 shearing and removal of the device anchor 122 from the patient's tissue 10).

FIGS. 1-5 show a depiction of the recapture and alignment steps used for chronic explanation. First a lasso 210 and a cinch/alignment tube 20 are used to reinstate a linkage between the implant 100 and a catheter-based explanation tool 1 (FIGS. 2-4). A protector cup 30 then ramps over the alignment cup 20 to center the chronically-implanted device 100 for subsequent shearing of the encapsulation 12 (FIG. 5; see FIGS. 6-10 for further details.) Note: A balance of cross-sectional, “real world”, and cut-away views are employed in this step-wise sequence (also in FIGS. 6-10) in an attempt to best highlight elements of the design concept.

FIG. 6 to FIG. 10 show a sequence continuing from the content presented in FIG. 5 that highlights key steps associated with shearing of surrounding encapsulation 12 (FIGS. 6-8) and the separation of the implant's anchoring tines 122 from the heart 10 (FIGS. 9-10). Further details associated with the shearing surfaces 52 design are shown in FIGS. 11-15. As can be seen in the plan view in FIG. 16 once the device 100 has been removed, remnants of the “seam ripped” encapsulation layer 12, 14 remain attached to the heart wall as dangling elements (the tine pathways 220 are shown as reference features).

Some elements in the shearing feature 50 are detailed in an end-on view of the catheter as shown in FIGS. 13-15 and further abstracted for clarity in FIGS. 11 and 12. In coordination with the alignment cup 20 at the end of the cinch tube the shearing surface 50 is aligned concentrically with the cross section of the implant's main body 100. As the protector cup 30 is pushed downward over the implant 100, the shearing surface 52 rides along the edge 110 of the implant 100. A series of gaps 80 may be included in the distal end 6 of the catheter's protector cup 30. These gaps 80 allow for the sheared encapsulation 14 to move out of the way once separated from the implant body 100. Adjustments between the inner diameter of the protector cup 30 and the cutting surface 52 as well as the height and number of these gaps 80 can be built into different embodiments to allow for differing clearances depending upon the thickness of the encapsulating tissue 12. Some embodiments might not even include orifices 80 but instead attach the shearing feature 50 toward the proximal end of the protector cup 30 and include longer-length contact sections 56 (on par with the length of the full device) wherein the sheared encapsulation 14 can remain within the protector cup 30 during the sequences outlined in FIGS. 6-10. Such an approach avoids any complications associated with sheared encapsulation 14 binding within an orifice, but may demand a slightly enlarged outer diameter for the protector cup to accommodate the sheared encapsulating tissue 14 during such device explanation processes. It would be expected that a single variant (or two) might be built that would best serve a majority of patients.

FIGS. 13-15 show an end-on view of the shearing feature 50 at the distal-end 6 of the catheter 1 with key side views shown in FIG. 14 (rib features) and FIG. 15 (expansion features). Bottom portions of FIGS. 14 and 15 remove clutter to show key elements. Around the perimeter of the shearing surface 50 the clearance gaps are broken to maintain a physical connection between the main body of the protector cup 30 and the front protective edge of the protector cup 30. These ribs 81 are detailed in FIG. 14 while a series of expansion features 70 are shown in FIG. 15. These expansion features 70 allow for a tight squeeze of the shearing surface 50 around the implant 100 perimeter while accommodating for different device sizing/tolerancing/alignment without motivating a binding response.

FIGS. 11 and 12 show a detail of some design elements associated with the cutting surface 52 of the shearing element 50 as pointed to in the cross-sectional call out of FIG. 13. The zoomed-in pictogram shows that the cutting surface 52 is slightly offset from the leadless pacer body 100 enabling removal of the tissue 10 from the implant's exterior 110 while also avoiding the likelihood of digging into the side of the pacer 110 and binding. The format of the shearing ring (gray) 50 forces the cutter in close proximity to the implant 100 using a built-in spring-force design.

Pushing the protector cup 30 and shearing element 50 down over the implant 100 can occur without having to instate added compression or tension on the myocardium 10 at the anchoring site. This effect can be accomplished through the use of a cinch/alignment tube 20 that is substantially rigid in coordination with a handle control element that moves the protector cup 30 and shearing element 50 relative to the fixed position cinch-alignment tube. Such actuation effectively, “seam-rips” the surrounding capsule, splaying it out to make the implant 100 readily accessible. With the bulk of the implant 100 body resident inside of the protector cup 30, the catheter then offers a stable counter balance to subsequently instated forces for tine 122 removal. As such, the tines 122 can be removed from the heart 10 without “reverse tenting” the heart's chamber wall, apex, or septum.

Retracting the catheter and the recaptured implant 100 complete the explanation procedure and leave the heart wall 10 accessible for other devices and does so without releasing fragments of the “seam ripped” capsule 14 into the patient's bloodstream.

Particularly, some objectives which are addressed by the disclosure are to:
offer support for the separation of leadless pacers 100 from surrounding physiologic encapsulation 12 stemming from chronic implantation, and/or
facilitate said support using a catheter-based system 1 that introduces minimal (and ideally no) use-based complexity beyond systems used for implantation and/or acute explanation.

The system may comprise one or more of the following features either alone or in any combination with each other:
a protected shearing surface 50 at the distal tip 6 of the explanation surface,
said shearing surface 50 enabling spring-based compression against the side of a leadless pacer 100 in a non-binding manner,
said non-binding faculty being supported by a series of strain relief features to accommodate for variances in the device 100 sizing and also a recurved cutting tip 50 that peels the sharpest leading edge 54 slightly away from the leadless pacer body 100,
protection of this shearing surface 50 through use of the leading edge 6 of the catheter's protector cup 30, adjoined to the main body of the protector cup 30 through a series of ribs 81,
said ribs enabling cutting even behind the protected zone to, in coordination with the main shearing edge 54 “seam rip” encapsulation surrounding the implant 100 and push removed encapsulation 14 out of the way through gaps 80 in the perimeter of the catheter's 1 distal tip ,
a means for reliably centering this shearing feature 50 about the recaptured implant 100 through a ramping element (i.e. the alignment cup 20) on the distal end of the cinch/alignment tube ,
a means for enabling shearing of the encapsulation 12 without instating unsafe tension or compression at the anchoring sight through the translation of the protector cup 30 relative to a fixed position cinch/alignment cup 20,
a means for presenting a stable counterforce at the anchor site to facilitate tine-based anchor removal without instating unsafe forces on the heart 10,
a means for segmenting the encapsulation capsule 14 such that it does not present an “empty sock” tube of tissue once the implant 100 has been removed (for example it may have three “wings” due to the three gaps 80 in the drawn embodiment between the main body of the protector cup 30 and its leading edge 6), and/or
a means for avoiding the freeing of capsule “debris” into freely circulating blood (i.e. leaving the remnants of the encapsulation capsule 14 attached to the heart).

A potential advantage(s) of the solution according to the present disclosure can at least be one of the following:
The catheter system 1 may be used as a means for removing any leadless system 100 if sized appropriately.
If an “old” device 100 can be removed, the “new” device 100 does not have to worry about possible problematic interactions, mechanical or otherwise, and the clinician accesses a much broader selection of available anchoring sites.
New patient populations may even become accessible with this offering in place as placement in younger patients, for example, becomes more palatable from the vantage of subsequent therapy support.

The features disclosed in regard with the system may also apply to a method for explanting an intracardiac pacing system and vice versa.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An apparatus for explanting an intracardiac medical device that is anchored to heart tissue of a patient, wherein heart tissue adheres to a casing the intracardiac medical device, wherein the apparatus comprises;
a protector device that extends along an extension direction, wherein the protector device is configured to surround the casing, when the protector device positioned at the casing,
an alignment device wherein the alignment device is configured to align the protector device and the casing with each other, when the alignment device engages the intracardiac medical device, and
a shearing element, wherein the shearing element is configured to move along the casing, when the protector device is moved along the casing in a moving direction towards a distal end of the intracardiac medical device, for shearing off heart tissue adhered to the casing.

2. The apparatus according to claim 1, wherein the shearing element comprises a cutting surface.

3. The apparatus according to claim 1, wherein the shearing element is positioned or positionable at a distal end of the protector device.

4. The apparatus according to claim 1, wherein the shearing element extends along a circumferential direction of the protector device.

5. The apparatus according to claim 1, wherein in a radial direction the shearing element is positioned inwards the protector device.

6. The apparatus according to claim 1, wherein the shearing element comprises a cutting edge, wherein the shearing element is configured such that the cutting edge is distant to the casing, when the protector device is moved along the casing in the moving direction.

7. The apparatus according to claim 1, wherein the apparatus is configured to press the shearing element against the casing, when the protector device is moved along the casing (in the moving direction.

8. The apparatus according to claim 1, wherein the shearing element is spring mounted at the protector device such that the shearing element presses against the casing, when the protector device is moved along the casing in the moving direction.

9. The apparatus according to claim 1, wherein the shearing element comprises at least one recess to increase a flexibility of the shearing element.

10. The apparatus according to claim 1, wherein the apparatus comprises at least one orifice configured to direct sheared-off heart tissue away from the casing.

11. The apparatus according to claim 10, wherein the apparatus comprises a cleavage element, configured to cleave sheared-off heart tissue along the moving direction, when the protector device is moved along the casing in the moving direction.

12. The apparatus according to claim 10 wherein the apparatus comprises a cleavage element, configured to guide the sheared-off heart tissue towards the at least one orifice.

13. The apparatus according to claim 1, wherein the shearing element is attached toward a proximal end of the protector device and includes contact sections, wherein the contact sections have a length equal to a length of the casing, wherein the sheared-off heart tissue remains within the protector device during explanation.

14. A method for explanting an intracardiac medical device that comprises a casing, a proximal end and a distal end comprising an anchor element, wherein the intracardiac medical device is anchored to a heart tissue of a patient via the anchor element, wherein heart tissue adheres to the casing, wherein an apparatus for explanting an intracardiac medical device according to claim 1 is provided, the method comprises the steps of:
engaging the intracardiac medical device with the alignment device such that the protector device are aligned with each other,
moving the protector device along the casing in a moving direction towards the distal end of the intracardiac medical device, and
shearing off heart tissue adhered to the casing.
